SEQUENCE LISTING (6 sequences)

SEQ ID NO: 1 - protein, 286 aa, Microbispora rosea
MSDLGITDDSVAQCKGLMLSICPDATIVDVCHTMTPWAVEEGARYIVDLPRFFPEGTVFATTTYPATGTEARSIAIRLKHAALGGARGQLAGSGSGFPRAEGAYIYVAPNNGLLTRVIEEHGYLEAYEVTSTDVIPAQPEPTFYSREMVAIPAAHLAAGYPLEKVGRKLSDDEIVRFDVKKPAASDGGLTGVVSAIDHPFGNVWTNVHRTDLEKLGVGYGTKLKITLEGVLPFELPLSPTFADAGEIGAPVAYLNSRGYLSLARNAASLAYPYNLKEGMSVKVTLA

SEQ ID NO: 2 - protein, 286 aa, Synthetic construct
MSDLGITDDSVAQCKGLMLSICPDATIVDVCHTMTPWAVEEGARYIVDLPRFFPEGTVFATTTYPATGTEARSIAIRLKHAALGGARGQLAGSGSGFPRAEGAYIYVAPNNGLLTRVIEEHGYLEAYEVTSTDVIPAQPEPTWYSREMVAYPAAHLAAGYPLEKVGRKLSDDEIVRFDVKKPAASDGGLTGVVSAIDHPFGNVWTNVHRTDLEKLGVGYGTKLKITLEGVLPFELPLSPTFADAGEIGAPVAYLNSRGYLSLARNAASLAYPYNLKEGMSVKVTLA

SEQ ID NO: 3 - protein, 286 aa, Synthetic construct
MSDLGITDDSVAQCKGLMLSICPDATIVDVCHTMTPWAVEEGARLIVDLPRFFPEGTVFATTSYPATGTEARSIAIRLKHAALGGARGQLAGSGSGFPRAEGAYIYVAPNNGLLTRVIEEHGYLEAYEVTSTDVIPAQPEPTWYSREMVAYPAAHLAAGYPLEKVGRKLSDDEIVRFDVKKPAASDGGLTGVVSAIDHPFGNVWTNVHRTDLEKLGVGYGTKLKITLEGVLPFELPLSPTFADAGEIGAPVAYLNSRGYLSLARNAASLAYPYNLKEGMSVKVTLA

SEQ ID NO: 4 - protein, 286 aa, Synthetic construct
MSDLGITDDSVAQCKGLMLSICPDATIVDVCHTMTPWAIEEGARYIVDLPRFFPEGTVFATTTYRATGTEARSIAIRLKHAALGGARGQLAGSGSGFPRAEGAYIYVAPNNGLLTRVIEEHGYLEAYEVTSTDVIPAQPEPTWYSREMVAYPAAHLAAGYPLEKVGRKLSDDEIVRFDVKKPAASDGGLTGVVSAIDHPFGNVWTNVHRTDLEKLGVGYGTKLKITLEGVLPFELPLSPTFADAGEIGAPVAYLNSRGYLSLARNAASLAYPYNLKEGMSVKVTLA

SEQ ID NO: 5 - protein, 286 aa, Synthetic construct
MSDLGITDDSVAQCKGLMLSICPDATIVDVCHTMTPWDVEEGARYIVDLPRFFPEGTVFATTTYPATGTEARSIAIRLKHAALGGARGQLAGSGSGFPRAEGAYIYVAPNNGLLTRVIEEHGYLEAYEVTSTDVIPAQPEPTWYSREMVAYPAAHIAAGYPLEKVGRKLSDDEIVRFDVKKPAASDGGLTGVVSAIDHPFGNVWTNVHRTDLEKLGVGYGTKLKITLEGVLPFELPLSPTFADAGEIGAPVAYLNSRGYLSLARNAASLAYPYNLKEGMSVKVTLA

SEQ ID NO: 6 - protein, 286 aa, Synthetic construct
MSDLGITDDSVAQCKGLMLSICPDATIVDVCHTMTPWAVEEGSRYIVDLPRFFPEGTVFATTTYPATGTEARSIAIRLKHAALGGARGQLAGSGSGFPRAEGAYIYVAPNNGLLTRVIEEHGYLEAYEVTSTDVIPAQPEPTWYSREMVAYPAAHLAAGYPLEKVGRKLSDDEIVRFDVKKPAASDGGLTGVVSTIDHPFGNVWTNVHRTDLEKLGVGYGTKLKITLEGVLPFELPLSPTFADAGEIGAPVAYLNSRGYLSLARNAASLAYPYNLKEGMSVKVTLA

(19) United States
(12) Patent Application Publication
(10) Pub. No.: US 2024/0035023 A1
(43) Pub. Date: Feb. 1, 2024

(54) COMPUTER IMPLEMENTED METHOD FOR ENGINEERING FLUORINASE ENZYMES FOR SYNTHESIS OF FLUOROPHENYL COMPOUNDS

(71) Applicant: Kcat Enzymatic Private Limited, Bengaluru (IN)

(72) Inventors: Pravin Kumar R, Bengaluru (IN); Gladstone Sigamani G, Bengaluru (IN); Roopa L, Bengaluru (IN); Naveen B K, Bengaluru (IN); Anuj J Shetty, Bengaluru (IN); Likith M, Bengaluru (IN)

(73) Assignee: Kcat Enzymatic Private Limited, Bengaluru (IN)

(21) Appl. No.: 18/340,913

(22) Filed: Jun. 26, 2023

(30) Foreign Application Priority Data
Jun. 24, 2022 (IN) 202241029679

(51) Int. Cl. C12N 15/10; G16C 20/64

(52) U.S. Cl. CPC C12N 15/1089; G16C 20/64; C12N 15/1058

(57) ABSTRACT

The present invention discloses a computer-implemented method for engineering fluorinase enzymes towards the synthesis of fluorophenyl compounds. Limited or no mechanistic details of fluorinase enzymes have hindered progress in understanding their catalytic mechanisms for synthesizing synthetic organofluorine compounds. Through a comprehensive computational screening process, specific methionine-sulfonium phenyl substrates, including [(3S)-3-amino-3-carboxypropyl][2,5-difluoro-4-(4-methoxy-2,4-dioxobutyl)phenyl]methylsulfonium, were designed and optimized using quantum chemical optimization techniques. This methodology uncovers crucial information on F— ion attack conformation and the catalytic mechanism of the substrate, leading to the formation of Methyl 3-oxo-4-(2,4,5-trifluorophenyl)butanoate. Furthermore, a protein sequence and 3D modeling-based enzyme screening process was employed to identify the most suitable enzyme for this substrate. The identified enzyme was then engineered using the mechanistic insights gained from the studies, resulting in improved substrate scope, stability and catalytic efficiency. This computer-based approach offers an efficient and precise alternative to traditional trial-and-error methods, advancing the field towards the successful synthesis fluorophenyl compounds.

### FIELD OF THE INVENTION

This invention relates to the field of Biology, Life Science, Computational Biology, Biocatalysis and Chemistry

### BACKGROUND OF INVENTION

Organofluorine chemistry has a significant impact on various aspects of everyday life and technology. The C—F bond is present in pharmaceuticals, agrochemicals, fluoropolymers, refrigerants, surfactants, anesthetics, material production, nutraceuticals, oil-repellents, and water-repellents, among other applications. Organofluorides constitute approximately 20% of registered pharmaceutical compounds since 1991 (Inoue M., et al., 2020), and about 16% of agrochemicals (Ogawa Y., et al., 2020). The strong binding nature of the C—F bond is highly desirable in developing industrial materials such as thermoplastics, elastomers, membranes, textile finishes, and coatings (Okazoe, T., 2009).

Several common APIs contain the fluorine (F—) ion, including Atorvastatin, known for reducing cholesterol and the associated risk of heart attack. Gefitinib is another molecule renowned for its anti-cancer properties, while Sitagliptin is a type 2 antidiabetic drug that lowers blood sugar levels in adults (FIG. 1). In these and many other compounds (˜45% of all active drugs), the F⁻ ion is directly attached to the aromatic ring, indicating the crucial role of the fluorinated phenyl group as an intermediate in synthesizing various significant organofluoride compounds. Chemical methods are typically employed to synthesize organofluorides under extreme conditions, using harmful reagents that require special techniques for handling fluorinating agents (Okazoe T., 2009). The challenges associated with chemical synthesis have increased the demand for reagents capable of selectively introducing F⁻ ion into organic compounds, particularly biological enzyme catalysts (Cheng, X. et al., 2021).

Enzymatic halogenation of organic compounds, including carbon-fluorine and carbon-chlorine bond formation, has been an active area of study. Enzymes such as fluorinases and chlorinases exhibit catalytic capabilities in this regard. Fluorinases, unlike chlorinases, possess an additional 21 amino acid region (AAKGGARGQWASGAGFERAEG) (Deng, H. et al., 2008). Among various enzyme-catalyzed synthesis methods, the direct formation of the C—F bond by fluorinase is the most effective and promising approach. Fluorinase can catalyze the synthesis of 5′-FDA from S-adenosyl-L-methionine (SAM), a natural substrate of the enzyme, and F— ion through nucleophilic attack, resulting in the formation of a C—F bond (FIG. 2) (Ma, L. et al., 2016). Consequently, fluorinase has become an essential biocatalyst for the synthesis of fluorinated nucleosides and their derivatives. Although fluorinase has been applied to catalyze non-natural substrates, it exhibits reduced catalytic activity for such substrates (Fraley and Sherman, 2018). Fluorinase is the sole biocatalyst capable of synthesizing compounds with C—F bonds, but its full potential remains largely unexplored. The low abundance and bioavailability of F— ions, coupled with their high heat of hydration, present challenges for achieving nucleophilic catalysis from water. Furthermore, the high electronegativity of F— ions limits an oxidation approach, suggesting that the physical properties of F— ions have restricted the evolution of F— ion biochemistry. The isolation of the Fluorinase enzyme in 2002 (O'Hagan, D., et al., 2002; Sananda, M. et al., 1986) marked the beginning of efforts to improve its activity. However, the binding site for F— ions has not been reported in any experimental structure (Sun, H., et al., 2016; Thompson, S., et al., 2016) (FIG. 3A). Plausible mechanisms of F— ion binding have been proposed (FIG. 3B), but information on a complex that could define the catalytic conformation using a synthetic substrate is lacking. Therefore, there is still much work to be done to engineer fluorinases for synthesizing organofluoride APIs. This is particularly important considering the hazards associated with the chemical synthesis of organofluorides, the limited sources of fluorinase, the scarcity of crystal structures (only nineteen to date), the low enzyme activity, the narrow substrate range, and the lack of systems that can compete with the corrosive hazardous chemical production of organofluorides (Cheng, X. et al., 2021).

### Prior Art

Aggarwal, V. K., Thompson, A., & Jones, R. V. (1994). Synthesis of sulfonium salts by sulfide alkylation; an alternative approach. Tetrahedron letters, 35(46), 8659-8660.
Cadicamo, C. D., Courtieu, J., Deng, H., Meddour, A., & O'Hagan, D. (2004). Enzymatic fluorination in Streptomyces cattleya takes place with an inversion of configuration consistent with an SN2 reaction mechanism. ChemBioChem, 5(5), 685-690.
Deng, H., O'Hagan, D., & Schaffrath, C. (2004). Fluorometabolite biosynthesis and the fluorinase from Streptomyces cattleya. Natural product reports, 21(6), 773-784. https://doi.org/10.1039/b415087mz
Inoue, M., Sumii, Y., & Shibata, N. (2020). Contribution of organofluorine compounds to pharmaceuticals. ACS omega, 5(19), 10633-10640.
Ma, L., Li, Y., Meng, L., Deng, H., Li, Y., Zhang, Q., & Diao, A. (2016). Biological fluorination from the sea: discovery of a SAM-dependent nucleophilic fluorinating enzyme from the marine-derived bacterium Streptomyces xinghaiensis NRRL B24674. RSC advances, 6(32), 27047-27051.
O'Hagan, D., Goss, R. J., Meddour, A., & Courtieu, J. (2003). Assay for the enantiomeric analysis of [2H1]-fluoroacetic acid: insight into the stereochemical course of fluorination during fluorometabolite biosynthesis in Streptomyces cattleya. Journal of the American Chemical Society, 125(2), 379-387.
O'Hagan, D., Schaffrath, C., Cobb, S. L., Hamilton, J. T. G. & Murphy, C. D. Biochemistry: biosynthesis of an organofluorine molecule. Nature 416, 279 (2002).
Ogawa, Y., Tokunaga, E., Kobayashi, O., Hirai, K., & Shibata, N. (2020). Current contributions of organofluorine compounds to the agrochemical industry. Iscience, 23(9), 101467.
Okazoe, T. (2009). Overview on the history of organofluorine chemistry from the viewpoint of material industry. Proceedings of the Japan Academy, Series B, 85(8), 276-289.
Raju, D. R., Kumar, A., Naveen, B. K., Shetty, A., Akshai, P. S., Kumar, R. P., . . . & Sigamani, G. (2022). Extensive modelling and quantum chemical study of sterol C-22 desaturase mechanism: A commercially important cytochrome P450 family. Catalysis Today, 397, 50-62.
Sanada, M. et al. Biosynthesis of fluorothreonine and fluoroacetic acid by the thienamycin producer, Streptomyces cattleya. J. Antibiot. (Tokyo) 39, 259-265 (1986)
Sergeev, M. E., Morgia, F., Javed, M. R., Doi, M., & Keng, P. Y. (2013). Enzymatic radiofluorination: Fluorinase accepts methylaza-analog of SAM as substrate for FDA synthesis. Journal of Molecular Catalysis B: Enzymatic, 97, 74-79.
Sun, H., Yeo, W. L., Lim, Y. H., Chew, X., Smith, D. J., Xue, B., & Ang, E. L. (2016). Directed evolution of a fluorinase for improved fluorination efficiency with a non-native substrate. Angewandte Chemie, 128(46), 14489-14492.
Thompson, S., McMahon, S. A., Naismith, J. H., & O'Hagan, D. (2016). Exploration of a potential difluoromethyl-nucleoside substrate with the fluorinase enzyme. Bioorganic Chemistry, 64, 37-41.

### Objects of the Invention

The objective of the present invention is to provide a computer-implemented method for engineering fluorinase enzymes towards the synthesis of fluorophenyl compounds.

By utilizing advanced modeling techniques and designing specific methionine-sulfonium phenyl substrates, the objective is to gain valuable insights into the catalytic binding mode of synthetic substrates and F— ion attack conformation, crucial for enzyme mechanism required in the synthesis of fluorophenyl compounds. The method aims to overcome challenges associated with traditional chemical synthesis methods that including environmental concerns and limited substrate selectivity of fluorinase enzymes.

Another objective is to employ modeling as a powerful tool in engineering fluorinase enzymes, enabling the rational design and optimization of enzyme structures. Through computational analysis and simulations within the active site of the enzyme, the objective is to enhance understanding of the underlying principles governing fluorinase catalysis, thereby guiding the synthesis of fluorophenyl compounds with improved efficiency and selectivity.

This approach holds the potential to revolutionize the field of fluorinase engineering by providing a systematic and efficient framework for enzyme optimization. By harnessing the power of computational modeling, this invention seeks to accelerate the development and commercialization of sustainable and scalable synthesis techniques for fluorophenyl compounds. The proposed method not only addresses the limitations of traditional approaches but also paves the way for the widespread industrial application of fluorophenyl compounds in sectors such as pharmaceuticals, agrochemicals, and materials.

### SUMMARY OF THE INVENTION

The Fluorinase enzyme was discovered in 2002 from a soil bacterium (O'Hagan, D., et. al., 2002, Sananda, M. et. al., 1986), and since then, scientists have been working on improving its activity. One of the important challenges is the enzyme's narrow substrate specificity and low stability (O'Hagan, D., et. al., 2003). The mechanism of Fluorinase, especially the binding site for F— ion, has not been reported in any experimental structure (Sun, H., et. al., 2016; Thompson, S., et. al., 2016). There is also a lack of information on a complex that could define a catalytic conformation using a synthetic substrate. Especially, where F— ion is in an attacking conformation against a substrate that could yield a fluorophenyl products. To address this, a methionine-sulfonium phenyl substrate was designed to fit into the active site of Fluorinase. The active site of Fluorinase, where the natural substrate binds, is quite voluminous. However, this voluminous structure cannot bind smaller phenyl substrates. Therefore, drug molecules were scanned (FIG. 4), and a trifluorophenyl moiety, used as an intermediate for the synthesis of sitagliptin, was chosen (FIG. 5). Based on this intermediate, a methionine-sulfonium phenyl substrate, A ([(3S)-3-amino-3-carboxypropyl][2,5-difluoro-4-(4-methoxy-2,4-dioxobutyl) phenyl] methyl sulfonium), was designed as a substrate (FIG. 6). Since there is limited information on the catalytic binding mode of F— ion and no information on the binding mode of F— ion against the methionine-sulfonium phenyl substrate, which is completely different from the natural substrate SAM, the following studies were carried out:

Extensive F— ion diffusion studies were conducted (FIG. 7), identifying a F-station in the active site that was completely desolvated and in a ready conformation for attaching the methionine-sulfonium phenyl substrate. Substrate of interest (mentioned above) was then modelled in the active site of Fluorinase, which had already been modelled with F-ion (FIG. 8). The active site, substrate of interest and the F— ion complex was optimized using DFT method, the altered substrate resulted in a different F— ion binding mode compared to previously reported studies. F— ion binding in the presence of the substrate was altered slightly from the native binding mode revealing a slightly different catalytic mechanism (FIG. 9 A, B). In the presence of the phenyl moiety the h-bonding interactions of F— ion with the catalytic residues, Ser145 and Thr67 was reduced, and F— ion showed closer interaction with the aromatic ring.

The main challenge was to achieve the precise conformation of the phenyl group within the active site of the enzyme. During the interaction between the phenyl group and the F— ion, there is a transfer of electron density from the phenyl group to the F— ion through the 71 electron system. As a result, the modelling of the phenyl moiety in the active site focused on facilitating π-π stacking interactions, which involve the overlap of electron clouds between aromatic rings. These interactions contribute to the stability and shape of the molecular system within the active site but do not directly interact with F— ion. Consequently, this arrangement leaves the C1 of the substrate available for F— ion to initiate an attack (FIG. 9 C, D)

In this study, QM/MM simulations were conducted over different near-attack conformations of the substrate until the reaction proceeded to form the product, trifluorophenyl moiety (as described in the FIG. 9 C). This complex, with a F— ion and a methionine-sulfonium phenyl substrate in the active site of Fluorinase that showed product formation in the QM/MM simulation and was used as the reference structure.

Further, a fluorinase enzyme demonstrating stable catalytic binding of the compound named, [(3S)-3-amino-3-carboxypropyl][2,5-difluoro-4-(4-methoxy-2,4-dioxobutyl)phenyl]methylsulfonium in the active site is identified among many fluorinases obtained from a non-redundant database, using a screening protocol that includes metadynamics simulations and free energy surface calculations to identify the most suitable fluorinase enzyme demonstrating stable catalytic binding of the substrate named in the active site. The selected fluorinase enzyme incorporates specific mutations derived using residue-residue contact maps to determine hydrophobic residues contributing to major physical contacts near the active site (FIG. 10) to optimize the binding affinity of the substrate, [(3S)-3-amino-3-carboxypropyl][2,5-difluoro-4-(4-methoxy-2,4-dioxobutyl)phenyl] methylsulfonium, producing an engineered enzyme with improved biocatalytic activity.

### BRIEF DESCRIPTION OF DRAWINGS

FIG. 1: Organofluoride compounds commonly found in the pharmaceutical, agricultural, and material science industries.

FIG. 2: A) Native reaction scheme catalyzed by the fluorinase enzyme, converting S-adenosyl-methionine (SAM) into 5′-Fluorodeoxyadenosine (FDA) with methionine as a by-product. B) Proposed reaction mechanism of the fluorinase enzyme, where the F— ion is bound to active site residues Ser145 and Thr67 through hydrogen bond interactions, facilitating its attack on the 5′ carbon adjacent to the sulfonium on the SAM molecule. This results in the formation of 5′-fluoro-deoxyadenosine with methionine as a by-product.

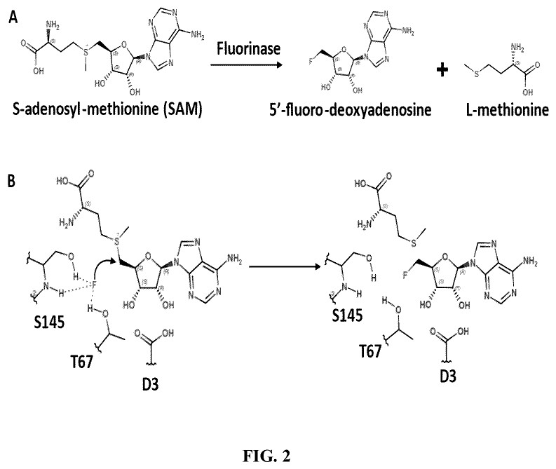

FIG. 3. The modelling of F— ion in the active site of fluorinase enzyme. A) The enzyme structure without the presence of the F— ion, showing the catalytic residues in a non-catalytic conformation. B) The entry of the F— ion modifies the enzyme's active site architecture, leading to interactions between Thr67 and Ser145 side chains and the F— ion, along with a hydrogen bond between Ser145 backbone nitrogen and the F— ion.

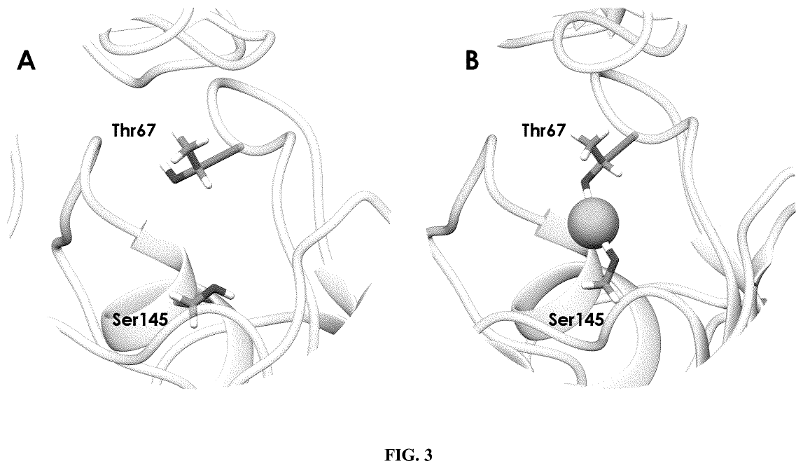

FIG. 4: The selected APIs feature a fluorophenyl moiety with attached methionine-sulfonium groups at the desired position, which can be fluorinated through enzymatic reaction with fluorinase. The APIs were truncated to fit within the active site, forming intermediates that can be utilized to generate the complete API. The engineered enzyme enables the attachment of the F— ion to these intermediates.

FIG. 5: Molecular modeling of F— ion and designed methionine sulfonium fluorophenyl substrates. A) Sitagliptin intermediate. B) Gefitinib precursor. C) Delafloxacin precursor. D) Enoxacin precursor in the active site of fluorinase. Distinct interactions were observed for each substrate. The sitagliptin intermediate displayed a superior binding conformation and interactions compared to the other substrates. The gefitinib precursor, delafloxacin precursor, and enoxacin precursor exhibited conformations with a limited number of clashes.

FIG. 6: Proposed reaction mechanism of fluorinase catalyzing the conversion of [(3S)-3-amino-3-carboxypropyl][2,5-difluoro-4-(4-methoxy-2,4-dioxobutyl)phenyl]methylsulfonium into Methyl 3-oxo-4-(2,4,5-trifluorophenyl)butanoate. The asterisk (*) indicates the transferred F— ion in the product, as inferred from the proposed reaction mechanism of the fluorinase enzyme.

FIG. 7: Free energy surface of F— ion diffusion derived from multiple simulation studies. The F— ion was initially positioned outside the active site and subjected to a bias force, allowing it to explore various low-energy gaussian wells along the translocation path. The amino acids along the path were identified as potential hotspots for enzyme engineering to facilitate the entry of F— ion. In the graph, blue regions represent low-energy states, while red indicates higher energy states. The yellow to red regions indicate barriers encountered during the translocation process.

FIG. 8: Modelling of the substrate [(3S)-3-amino-3-carboxypropyl][2,5-difluoro-4-(4-methoxy-2,4-dioxobutyl)phenyl]methylsulfonium within the active site of the fluorinase enzyme, highlighting the catalytic residues in cyan sticks and the substrate in grey sticks. This arrangement exposes the C1 atom (highlighted as an orange ball) of the substrate, providing a suitable position for the F— ion to initiate an attack.

FIG. 9: A) S-adenosyl methionine (SAM) (magenta sticks) and B) Substrate of interest, [(3S)-3-amino-3-carboxypropyl][2,5-difluoro-4-(4-methoxy-2,4-dioxobutyl)phenyl]methylsulfonium (grey sticks), were modeled within the active site of the fluorinase enzyme using quantum chemical optimization with DFT. F— ion was also incorporated into the optimized binding conformations. It was observed that the binding mode of the substrate of interest differs from that of SAM, where SAM is the native substrate for the fluorinase enzyme. C) The relative orientation of the F— ion attack conformation with respect to the π orbitals of the phenyl moiety is crucial in determining the fluorophenyl product. D) Anionic pi interaction between F— ion and the phenyl ring, where F— ion is attracted towards the ring, plays a significant role in the engineering process.

FIG. 10. The residue-residue contact map of fluorinase enzyme which depicts regions of high residue-residue contacts indicating strong physical interactions between the residues in x-axis vs residues in y axis. The square box on the graph depicts residue with low pLDDT value in the region of higher contacts, lower pLDDT may correlate with the structural stability associated with specific mutations. These residues are chosen as hotspot for engineering the enzyme.

FIG. 11: Computational method for engineering fluorinase enzyme. The method consists of three major steps. A) Modelling F— ion and the methionine-sulfonium phenyl substrate within the active site of fluorinase enzyme to simulate a specific F— ion attack conformation and generate a reference complex with a catalytic conformation (Brown colored boxes). This reference complex serves as a template for B) identifying a fluorinase enzyme with optimal binding affinity for the selected methionine-sulfonium phenyl substrate (Blue boxes). C) The method further includes a process for engineering the enzyme to enhance substrate affinity (Green boxes).

### DETAILED DESCRIPTION OF THE INVENTION

### Terminologies Explained/Abbreviations

### Computer Implemented Method

“Computer Implemented Method” refers to methods or processes that are implemented using computer technology; in the present context there are several advantages over other methods of problem-solving such as (1) Speed and Efficiency: processing vast amounts of data and executing complex calculations at high speeds and is particularly valuable as the data is computationally intensive and would be time-consuming or practically infeasible to solve manually, (2) Scalability: efficiently handle large datasets, process numerous iterations, providing scalability that cannot be achieved manually, (3) Automation and Repetition: for tasks such as data analysis, simulations, optimization, and iterative processes, (4) Storage and Retrieval: store large datasets, previous results, and reference materials for quick access and analysis; allows for more comprehensive problem-solving by leveraging previously processed information and facilitating data-driven decision-making, (5) Visualization and Interaction: powerful visualization capabilities, allowing users to represent complex data in meaningful ways. Visualization aids in understanding patterns, relationships, and trends within the data, leading to better insights and decision-making. Additionally, computers enable interactive problem-solving through user interfaces, where users can input data, modify parameters, and observe the immediate impact on the results, (6) Iterative Refinement: iterative process facilitates experimentation and exploration of various scenarios, enabling better optimization and improvement of the problem-solving approach.

### Simulation

“Simulation” refers to the process of using a model to imitate and study the behavior of a real process. In the present context it is used to understand the behaviour of a fluorinase enzyme system which has F— ion and a substrate in the active site. The advantages of simulating such a system includes (1) Cost and Time Efficiency: Simulations allow for rapid and cost-effective exploration of different scenarios and designs without the need for extensive resources, (2) Complexity Handling: Simulations are particularly advantageous when dealing with complex systems or phenomena that are difficult to analyze mathematically or solve analytically. By using computational models, simulations can represent and study intricate relationships, interactions, and behaviors of complex systems. F— ion biochemistry is one such phenomena, (3) Parameter Exploration and Sensitivity Analysis: Simulations enable the exploration of a wide range of parameters and their effects on the system being modelled. Researchers can analyze how changes in variables impact the overall behaviours, performance, or outcomes of the system, (4) Optimization and Design: Simulations support optimization by allowing researchers and engineers to test different design alternatives, configurations, or strategies. In the present context, it was possible to evaluate the performance of various options, identify bottlenecks, and optimize the system's behavior or efficiency, (5) Data Generation and Analysis: Simulations generate large amounts of data that can be analyzed to gain insights and inform decision-making. In the present context, it was possible to analyze the output of simulations to identify patterns, correlations, or anomalies within the simulated system. This data-driven approach enhances understanding and facilitates decision-making.

### Methionine Sulfonium Salts

“Methionine Sulfonium Salts” refers to compounds which contain a tricoordinate sulfur atom bearing a positive charge on sulfur are called sulfonium salts and that which is attached to methionine is called methionine sulfonium salts. In the present context such a moiety is crucial for activity of fluorinase enzyme. The enzyme has no activity against S-adenosyl-homocysteine (SAH), the non-sulfonium analogue of SAM, which is a natural substrate of fluroniase (Sergeev, M. E., et. al., 2013). Therefore, methionine sulfonium moieties are a logical starting point to explore when expanding the substrate scope of fluorinase. Several methods to synthesize sulfonium salts have been described previously, (Aggarwal, V. K. et. al., 1994, Sander, K. et. al., 2015) are adopted to synthesize the methionine sulfonium salts required for studying the substrate scope of the engineered fluorinase described in this embodiment.

### Wild or Wild-Type

The term “wild” or “wild-type” refers to a polypeptide sequence naturally occurring within an organism and can be procured from a source found in nature.

### Mutagenesis

The term “Mutagenesis” refers as changing the function of protein by introducing a mutation on a specific position of the protein. For instance, the natural phenylalanine at position 143 has been changed to tryptophan, this process by which incorporating different amino acid into a protein by mutating a position is known as mutagenesis.

### Molecular Dynamics

“Molecular dynamics” is a computational simulation method derived from Newtonian physics, used to study the dynamic behavior and movement of atoms and molecules over time. It models the physical interactions between individual particles, considering forces such as electrostatic interactions, van der Waals forces, and bond stretching. By numerically integrating the equations of motion derived from Newton's laws, molecular dynamics simulations provide valuable insights into the structural changes, thermodynamic properties, and dynamic processes of molecular systems. Typically, molecular dynamics simulations consist of multiple steps such as, Energy minimization, NVT (Equilibration of system by maintaining constant volume and temperature of the system), NPT (Equilibration of system by maintaining constant pressure)

### Metadynamics

“Metadynamics” is an extension to the traditional molecular dynamic simulations designed to explore the properties of multidimensional free energy surfaces (FES) in complex many-body systems, wherein a common approach involves employing coarse-grained non-Markovian dynamics within a reduced space defined by a small set of collective variables. These dynamics exhibit a distinctive attribute, a history-dependent potential term, that gradually fills the minima in the FES over time. This unique characteristic enables efficient exploration and precise determination of the FES with respect to the collective variables.

### Collective Variables

In this context, the term “Collective Variables” or “CV” refers to set of atoms or a group of atomic coordinates of amino acids used to study metadynamics simulations. The CV plays an important role in metadynamics where the bias potential applies directly to CV atoms or coordinates. The applied bias potential identifies different gaussian wells or bins throughout the simulations over the time.

### Trajectory

A “trajectory” is represented as a series of coordinates or states across the simulation time, allowing the visualization and analysis of the object's or system's motion.

### Quantum Mechanics/Molecular Mechanics (QM/MM)

“Quantum Mechanics/Molecular Mechanics (QM/MM)” is a hybrid sampling approach that incorporates quantum mechanical calculations simulations to a set number of atoms in the study and applies molecular mechanics terms to the remaining atoms in the system. Studying the biochemical system at the electronic and subatomic level is computationally expensive, on the other hand, the accuracy of molecular mechanics is limited to the atom level, which makes it difficult to understand the transition level events that are rate limiting steps in a reaction. The hybrid approach of QM/MM results in a method that computationally allows for studying reaction sites at the atomic level and the rest of the system at a molecular level by defining a QM-MM boundary condition that separates the Quantum chemical calculation region and the regions considered under molecular mechanics terms.

### Gaussian Accelerated Molecular Dynamics (GaMD)

“Gaussian accelerated Molecular Dynamics (GaMD)” is an extension to conventional molecular dynamics simulation wherein exploration of conformational transitions across the potential energy landscape of the system is achieved through the application of a harmonic boost potential that follows a Gaussian distribution. In this context GaMD is used to study F-ion entry into the active site.

### The General Atomic and Molecular Electronic Structure System

“The General Atomic and Molecular Electronic Structure System (GAMESS)” is a widely used electronic structure software package for computational chemistry. It provides ab initio quantum chemistry calculations, density functional theory calculations, quantum mechanics/molecular mechanics (QM/MM) calculations, and other semi-empirical calculations.

### Density Functional Theory (DFT)

The term “density functional theory (DFT)” is a computational quantum mechanical modelling technique that helps in studying the electronic structure and characteristics of atoms, molecules, and solids.

### AlphaFold

“AlphaFold” is a convolutional neural network (CNN)-based deep learning program by DeepMind that predicts protein structures with great accuracy based on their amino acid sequences.

pLDDT

“pLDDT” is a per-residue predicted confidence score to determine the confidence and accuracy of prediction of a modelled residue. The predicted confidence score is based on the local distance difference test (LDDT) that is a superimposition free measure of the atoms-atom distances in a modelled structure to validate the accuracy of the structure. The pLDDT confidence score ranges from 0-100, with greater than 90 being expected to be a residue modelled with high accuracy. In this context, low pLDDT means any value lesser than or equal to 75. Low pLDDT score residues were considered as hotspots to be mutated into residues with higher pLDDT score, which in turn indicates a greater confidence in the 3D structure of the protein.

### Substrate Binding Affinity

“Substrate binding affinity” refers to the degree of interaction between a substrate molecule and the binding site on an enzyme or receptor is referred to as substrate binding affinity. It influences the effectiveness of enzymatic reactions. In this context refers to the favourable interaction between substrate and active site resides of the enzyme. Better binding affinity is where the steric clashes are minimum.

### Hotspots

The “hotspots” are specific amino acid positions on a polypeptide that are chosen after analysis for mutations which can bring about a change in the functional properties of the polypeptide.

### Contact Score or Contact Map

The terms “contact score” or “contact map” in this context refers to a method of ranking interactions that evaluates residue-residue interaction as a function of distance and physical van der Waal's contacts. Higher contact score indicates greater physical contacts of a residue with the target substrate or residue.

### Free Energy Surface (FES) Graph or Plot

“Free energy surface (FES) graph or plot” refers to a method of visualizing the output of the metadynamics simulation as a function of the collective variables defined for the experiments. The Collective variables are defined in the x and y axes and the resulting surface is coloured based on the potential energy of the system under study. For the purposes of this embodiment, deeper potential wells and potential wells closer to the origin of the FES graph are considered to be an improvement over the reference FES graph.

### Favourable and Unfavourable Interactions

In this context, interactions, both favourable and unfavourable, are those interactions that are contributed by the residues in the active site. Favourable interactions refer to those interactions in the environment of the enzyme or protein that can facilitate stronger binding of the target molecule, be it a substrate or residue. Interactions that are favourable are charged electrostatics interactions, hydrogen-bonding interactions, hydrophobic interactions. Unfavourable clashes are those interactions that are caused by overlapping van der Waal's radii. Unfavourable clashes tend force the substrate in an unrealistic or stressed conformation which can be considered as a high energy state. Minimising these high energy states and increasing stronger binding interactions leads to the substrate attaining a better binding mode in the active site of the enzyme.

### Induced Fit Modes

“Induced fit modes” in this context refers to a method of structurally modelling the substrate into the active site of an enzyme by using ab initio methods to fit the substrate into the active site of generated ensembles of the enzyme active site structure.

### Percent Identity or Percentage Identical

In this context, the term “percent identity” or “percentage identical” are used to describe comparisons between polypeptides. To obtain this percentage, two sequences are optimally aligned over a comparison window, which may include gaps (i.e., deletions or additions) in the polypeptide sequence compared to the reference sequence, which does not contain gaps. The percentage is calculated by counting the number of positions in which the same nucleic acid base or amino acid residue appears in both sequences, dividing the number of matched positions by the total number of positions in the comparison window, and multiplying the result by 100 to obtain the percentage of sequence identity.

### Acidic, Basic, Polar, Non-Polar Amino Acids

The acidic amino acids or residues include L-Glu (E) and L-Asp (D), basic amino acids or residues include L-Arg (R) and L-Lys (K), polar amino acids or residues include L-Asn (N), L-Gln (Q), L-Ser (S) and L-Thr (T), non-polar amino acids or residues include L-Gly (G), L-Leu (L), L-Val (V), L-Ile (I), L-Met (M) and L-Ala (A)

### Hydrophilic, Hydrophobic, Aromatic, Aliphatic Amino Acids

hydrophilic amino acids or residues include L-Thr (T), L-Ser (S), L-His (H), L-Glu (E), L-Asn (N), L-Gln (Q), L-Asp (D), L-Lys (K) and L-Arg (R), hydrophobic amino acids or residues include L-Pro (P), L-Ile (I), L-Phe (F), L-Val (V), L-Leu (L), L-Trp (W), L-Met (M), L-Ala (A) and L-Tyr (Y), aromatic amino acids or residues include L-Phe (F), L-Tyr (Y) and L-Trp (W) and aliphatic amino acids or residues include L-Ala (A), L-Val (V), L-Leu (L) and L-Ile (I). Although owing to the pKa of its heteroaromatic nitrogen atom L-His (H) it is sometimes classified as a basic residue, or as an aromatic residue as its side chain includes a heteroaromatic ring.

### Amino Acid Difference or Residue Difference

A “Amino acid difference or residue difference” refers to a change in the residue at a specified position of a polypeptide sequence when compared to a reference sequence. For example, a residue difference at position X116, where the reference sequence has a phenylalanine, refers to a change of the residue at position X116 to any residue other than phenylalanine. As disclosed herein, an enzyme can include one or more residue differences relative to a reference sequence, where multiple residue differences typically are indicated by a list of the specified positions where changes are made relative to the reference sequence.

### Reference Sequence

“Reference sequence” refers to a defined sequence to which another (e.g., altered) sequence is compared. In this context the reference sequence is Fluorinase from Streptomyces cattleya (Accession no. Q70GK9.1, PDB ID: 5FIU)

### Conservative Amino Acid Substitutions or Mutations

“Conservative amino acid substitutions or mutations” refer to the interchangeability of residues having similar side chains, and thus typically involves substitution of the amino acid in the polypeptide with amino acids within the same or similar defined class of amino acids.

### Non-Conservative Substitution

“Non-conservative substitution” refers to substitution or mutation of an amino acid in the polypeptide with an amino acid with significantly differing side chain properties.

### Methodology

The engineered flourinases used to synthesize the trifluorophenyl compounds are designed computationally as described below.

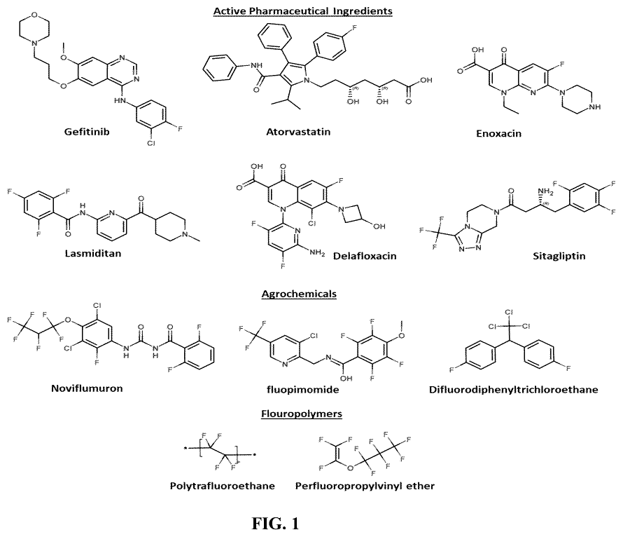

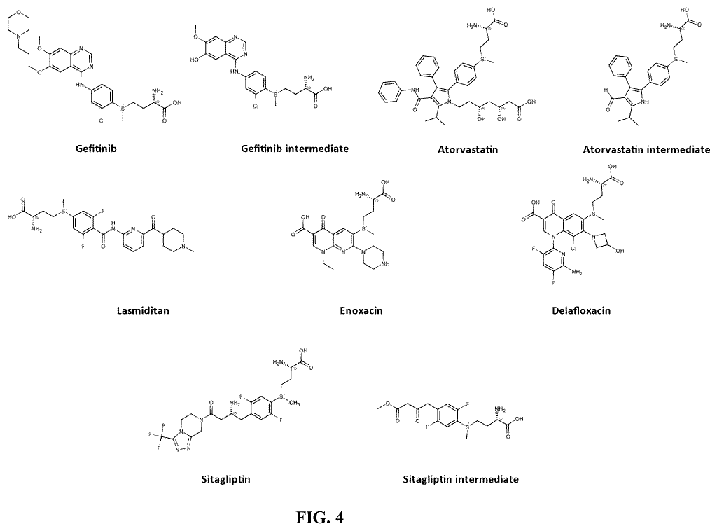

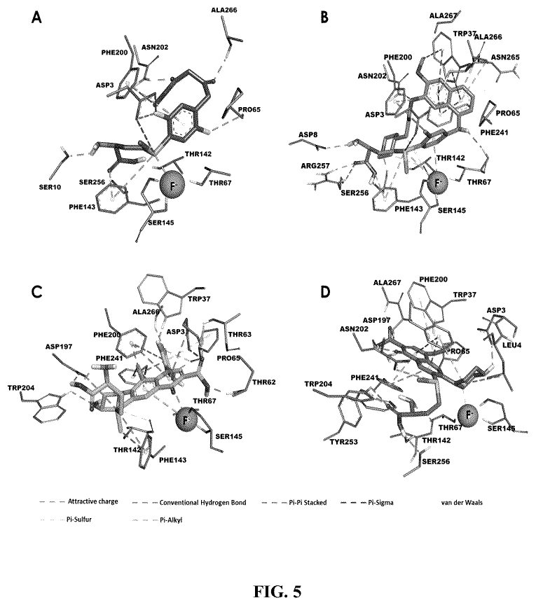

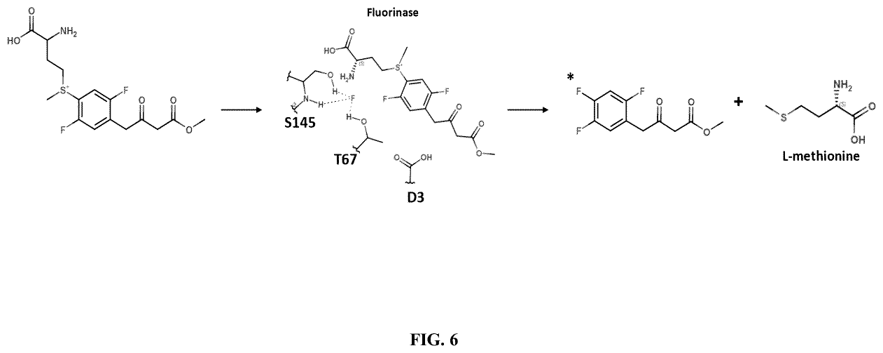

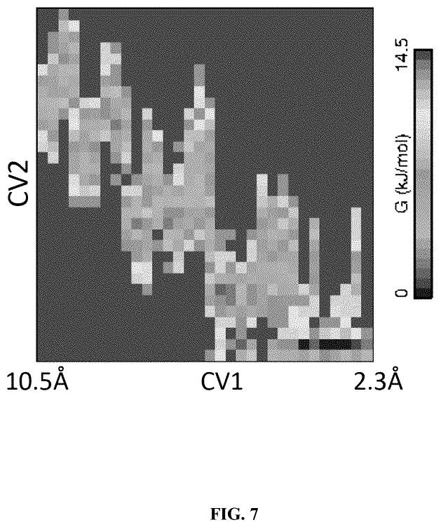

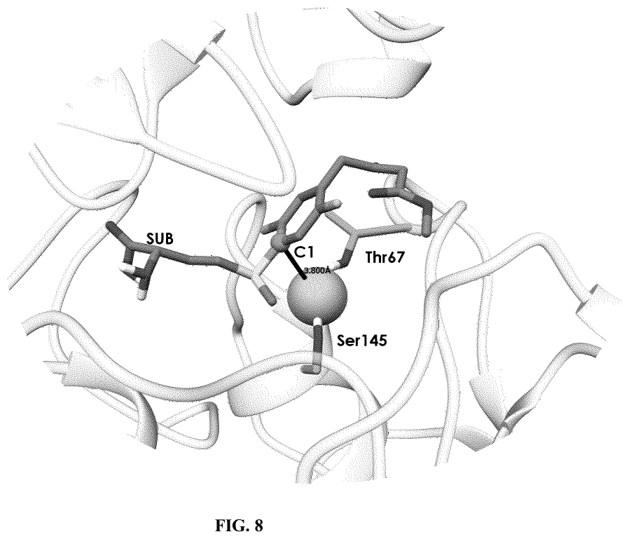

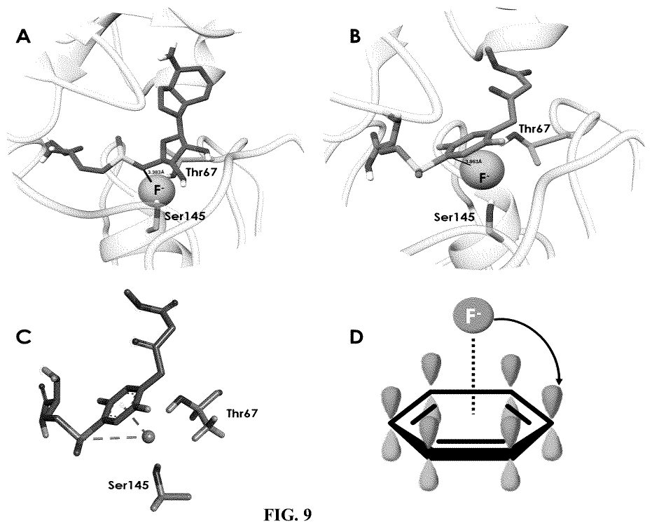

1 Generation of Reference Enzyme-Substrate Complex:
1.1 Fluorinase from Streptomyces cattleya (Accession no. Q70GK9.1, PDB ID: 5FIU) was selected to develop a reference enzyme-substrate model.
1.2 To model the F⁻ ion into the active site of the enzyme and understand its diffusion path. a Gaussian accelerated Molecular Dynamics (GaMD) approach was employed. GaMD enables enhanced sampling and free energy calculations. allowing for an exploration of the pathway and energetics associated with the F ion entering the enzyme's active site.
1.2.1 Using GaMD simulations, the diffusion of the F⁻ ion was studied, providing insights into the conformational changes of the active site necessary for the ion to reach the catalytic site of the active site. The simulations sampled a wide range of conformational space. allowing for a comprehensive exploration of the potential energy landscape.
1.2.2 Through the GaMD simulations, the least energy conformation of the F⁻ ion in the active site was obtained. This conformation represents the stable binding mode of the enzyme-F⁻ complex. The simulation revealed a conformational transition within the enzyme, enabling the formation of a stable catalytic attack conformation.
1.3 The enzyme-F⁻ ion complex obtained previously served as the reference model for subsequent substrate modelling. The specific substrate used was methionine-sulfonium phenyl substrate, denoted as [(3S)-3-amino-3-carboxypropyl][2,5-difluoro-4-(4-methoxy-2,4-dioxobutyl)phenyl]methylsulfonium.
1.3.1 Designing a specific methionine-sulfonium phenyl substrate, [(3S)-3-amino-3-carboxypropyl][2,5-difluoro-4-(4-methoxy-2,4-dioxobutyl)phenyl]methylsulfonium. The active site complexed with S-adenosylmethionine (SAM) revealed a large pocket; however, our specific focus was on active pharmaceutical ingredients (APIs) that contained a fluorophenyl moiety (FIG. 1). To address this, we followed the method as described below. We collected APIs with a fluorophenyl moiety from relevant literature sources and introduced methionine-sulfonium groups at specific positions of interest (FIG. 4). This modification aimed to convert the fluorophenyl moiety of the identified APIs into corresponding substrates. Subsequently, these substrates underwent 3D optimization to refine their conformations. To ensure compatibility with the active site, the APIs were appropriately truncated, forming intermediates that could be used to generate the complete API. The attachment of F ion to these intermediates could be achieved using the engineered enzyme. Subsequent modelling studies were performed within the active site of the fluorinase enzyme to evaluate and identify the optimal substrate from the various modified variations (FIG. 5). FIG. 6 illustrates a plausible reaction mechanism mediated by the fluorinase enzyme for the catalysis of [(3S)-3-amino-3-carboxypropyl][2.5-difluoro-4-(4-methoxy-2,4-dioxobutyl)phenyl]methylsulfonium to into Methyl 3-oxo-4-(2,4,5-trifluorophenyl)butanoate. The proposed mechanism outlines the steps involved in the conversion process. Additionally, in FIG. 7, the path taken by fluoride ions to achieve the attack conformation is depicted. The diagram showcases the journey of fluoride ions toward the catalytic center, wherein the necessary configuration for initiating the attack conformation is established. The details of the modelling studies are described below
1.3.2 To model the [(3S)-3-amino-3-carboxypropyl][2,5-difluoro-4-(4-methoxy-2,4-dioxobutyl)phenyl]methylsulfonium, within the active site, an initial conformation was generated where the methylsulfonium moiety retained the same conformation as observed in the crystal structure (FIG. 8). The remaining part of the substrate was positioned in a manner that the phenyl group faced away from the active site. allowing the C1 orbital of the substrate to be oriented towards the F⁻ ion. This orientation facilitated a favourable attack conformation between the substrate and the F⁻ ion.
1.3.3 The active site residues were extracted from the enzyme structure. Both the active site complex with F⁻ ion and substrate structures were optimized using quantum chemical calculations to obtain accurate electronic distributions and molecular orbitals.
1.3.4 Quantum chemical calculations were performed using the GAMESS software to determine the electronic structure. Density functional theory (DFT) was employed to calculate the molecular orbitals and their corresponding energies of the enzyme and substrate complex.
1.3.5 The orbital energies and distributions obtained from the calculations were analysed to identify important interactions between the substrate and the active site residues.
1.3.6 Based on the molecular orbital analysis, the reaction coordinates representing the attack conformation of P ion towards the substrate were extracted.
1.3.7 The reaction coordinates obtained from the quantum chemical calculations of the active site, F ion, and substrate complex were used as a reference for 3D coordinate transformation (FIG. 9B). The coordinates of the F⁻ ion and the substrate were transformed into the newly modelled fluorinases.
1.3.8 The coordinate transformation was performed using 3D geometric matching. specifically utilizing the backbone atoms of the residues within the active site. The coordinates of the residues Asp3, Tyr64, F⁻ ion, and substrate from the reference complex were transferred into the active site of the newly modelled fluorinases. This process ensured an accurate alignment of the atoms and preserved the relative positions and orientations of the components within the active site.

2 Identification of a Fluorinase Enzyme with Optimal Binding Affinity for the Substrate, [(3S)-3-amino-3-carboxypropyl][2,5-difluoro-4-(4-methoxy-2,4-dioxobutyl)phenyl]methylsulfonium.
2.1 The fluorinase protein sequences were retrieved from a non-redundant database using keyword searches for “chlorinase,” “fluorinase,” and “halogenase.” These keywords were chosen to specifically target enzymes involved in halogenation reactions.
2.2 A curation process was conducted to filter and extract only the fluorinase sequences using the specific sequence pattern “AAKGGARGQWASGAGFERAEG,” which serves as a unique fingerprint for fluorinases. Multiple sequence alignment was performed, comparing sequences with and without the identified pattern.
2.3 The obtained fluorinase protein sequences were modelled using the tool AlphaFold. The active site residues were located, and the coordinates relevant for the reaction, the F⁻ ion, substrate, and relevant residues were transformed using the same methodology described in section 1.3.8.
2.4 The newly modelled fluorinase structures, with the incorporated F⁻ ion and substrate, underwent a screening protocol that involved metadynamics simulations and free energy surface calculations.
2.5 The collective variables (CVs) used in the metadynamics simulations were the distance between the center of mass (COM) of Ser145 and Thr67 residues and the F⁻ ion, as well as the distance between the C1 atom of the substrate and the F⁻ ion.
2.6 The resulting free energy surface graph was processed using an image processing method to identify the best minima, represented as Gaussian wells. These minima corresponded to configurations where the catalytic residues, F⁻ ion, and substrate exhibited the closest possible interactions.
2.7 The goal was to identify the most suitable fluorinase enzyme that demonstrated stable catalytic binding of the specific substrate named [(3S)-3-amino-3-carboxypropyl][2,5-difluoro-4-(4-methoxy-2.4-dioxobutyl)phenyl]methylsulfonium within its active site. based on the analysis of the obtained free energy surface.

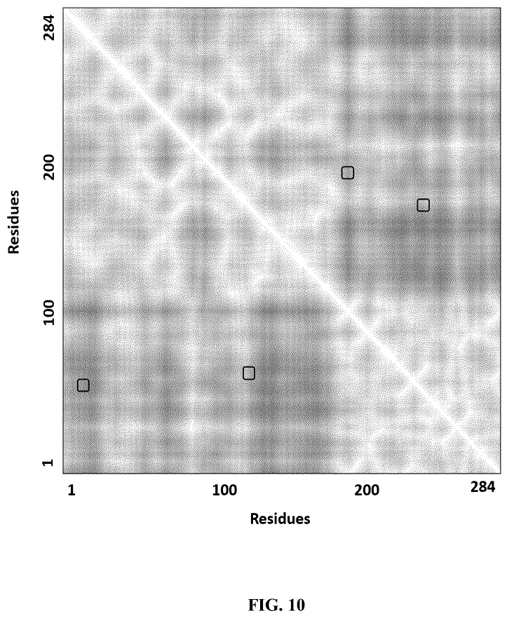

3 Engineering of a Fluorinase Enzyme to Enhance Substrate Binding Affinity for [(3S)-3-amino-3-carboxypropyl][2,5-difluoro-4-(4-methoxy-2,4-dioxobutyl)phenyl] methylsulfonium
3.1 QM/MM simulations were performed on the selected enzyme complexed with P ion and the substrate to investigate the reaction dynamics. The reaction coordinates were extracted at different stages of the reaction for further analysis.
3.2 Residue contact maps were generated using the extracted coordinates to identify regions with higher contact frequencies. particularly within 7 Å from the active site. The pLDDT values were extracted from the protein modelling studies, and the residues with lower pLDDT values were identified as hotspots. Hydrophobic residues were selected as substitution candidates, and mutations were introduced accordingly. A total of 1000 variants were generated through mutation steps (FIG. 10).
3.3 Following the mutation steps outlined in section 2.1 to 2.7, the screening protocol was applied to identify the best enzyme variant with improved binding affinity for the substrate.
3.4 The engineered fluorinases provided here have one or more improved properties in converting the synthetic substrates mentioned in this embodiment to the product which is not naturally occurring in any wild type fluorinase enzymes of any organisms. The engineered fluorinase polypeptide comprises of an amino acid sequence that is at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 2, 3, 4, 5, and 6 where X143 is W, X151 is Y, X63 is S, and X65 is R.
3.5 In some embodiments of an engineered fluorinase of the disclosure, the amino acid residues at a residue position can be defined in terms of the amino acid “features” (e.g., type or property of amino acids) that can appear at that position. Thus, in some embodiments the amino acid residues at the positions specified above can be selected from the following features: X38 is a Polar, charged, aliphatic or aromatic residue; X39 is an Aliphatic or polar residue; X43 is a Polar, charged, or aliphatic residue; X45 is a Polar, charged, aliphatic or aromatic residue; X63 is a non-polar or aliphatic residue; X65 is a non-polar or aliphatic residue; X156 is an aliphatic residue; X195 is a Polar, charged, aliphatic or aromatic residue

The mutations on the engineered fluorinases are given in Table 1.

TABLE 1
Mutations on Engineered Fluorinases

| Sequence | |
|---|---|
| ID | Mutations |
| 2 | PHE143TRP_ILE151TYR |
| 3 | TYR45LEU_THR63SER_PHE143TRP_ILE151TYR |
| 4 | VAL39ILE_PRO65ARG_PHE143TRP_ILE151TYR |
| 5 | ALA38ASP_PHE143TRP_ILE151TYR_LEU156ILE |
| 6 | ALA43SER_PHE143TRP_ILE151TYR_A195THR |

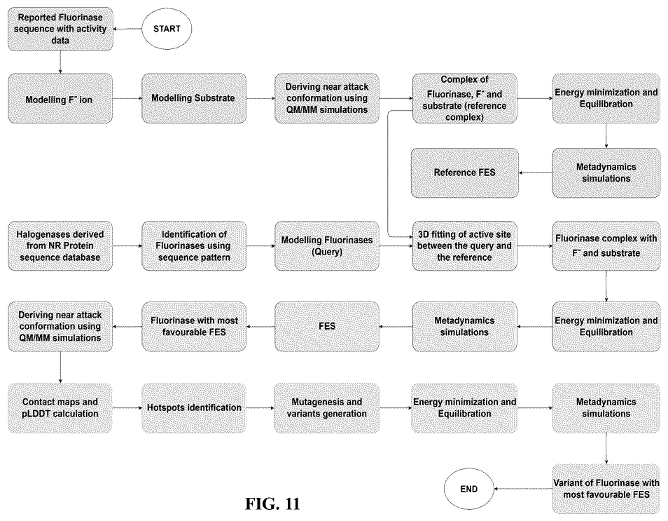

The entire above process from section 1 to 3 is depicted as a process diagram in FIG. 11

### Advantages/Significance of the Invention

The disclosed invention provides a pioneering computer-implemented method for engineering fluorinase enzymes towards the synthesis of fluorophenyl compounds. By leveraging computational modeling, the method offers advantages in terms of efficiency, overcoming challenges of chemical synthesis, expanding substrate scope, rational enzyme design. The approach represents a significant advancement in fluorinase engineering and holds immense potential for widespread industrial use of fluorophenyl compounds. The key advantages are listed here;

Enhanced Efficiency: By designing specific substrates and conducting modeling studies, the method accelerates the identification of optimal enzyme-substrate interactions, leading to more efficient catalytic activity and synthesis of fluorophenyl compounds.

Overcome Challenges of Chemical Synthesis: Traditional chemical synthesis methods for organofluorine compounds often pose environmental concerns and encounter stability issues. By employing this computer-implemented method, the challenges associated with chemical synthesis are addressed, enabling a more sustainable and environmentally friendly approach to fluorophenyl compound production.

Expanded Substrate Scope: The method's focus on engineering fluorinase enzymes allows for the expansion of substrate scope. Through computational modeling and substrate design, the method facilitates the synthesis of a wide range of fluorophenyl compounds, opening doors to various sectors such as pharmaceuticals, agrochemicals, and materials science.

Enzyme Design: The integration of computational modeling enables a rational and targeted approach to enzyme design and optimization. By gaining valuable insights into catalytic binding modes and F— ion attack conformations, the method enables the selection and modification of fluorinase enzymes to enhance their activity and substrate selectivity, resulting in more effective synthesis of fluorophenyl compounds.

Scalable Industrial Applications: The improved stability, substrate scope, and catalytic activity of the engineered fluorinase enzymes make large-scale production of fluorophenyl compounds feasible. This method paves the way for scalable and commercially viable production processes, benefiting industries such as pharmaceuticals, agrochemicals, and materials science.

What is claimed is:

1. A computer-implemented method for engineering a fluorinase enzyme for the synthesis of fluorophenyl compounds, the method comprising steps:
Step 1. Designing a methionine-sulfonium phenyl substrate, by:
a. Identifying active pharmaceutical ingredients (APIs) containing a fluorophenyl moiety;
b. Introducing a methionine-sulfonium group at a position of interest to convert the fluorophenyl moiety of the identified APIs into respective substrates; and
c. Conducting modeling studies of the converted substrates within the active site of the fluorinase enzyme to determine the optimal substrate,
d. Optimal substrate derived is [(3S)-3-amino-3-carboxypropyl][2,5-difluoro-4-(4-methoxy-2,4-dioxobutyl)phenyl]methylsulfonium.
Step 2. Performing three-dimensional (3D) modeling of a F⁻ ion and the methionine-sulfonium phenyl substrate, ([(3S)-3-amino-3-carboxypropyl][2,5-difluoro-4-(4-methoxy-2,4-dioxobutyl)phenyl]methylsulfonium), within the active site of the fluorinase enzyme to simulate a specific F⁻ ion attack conformation.

2. The method of claim 1, wherein a fluorinase enzyme demonstrating stable catalytic binding of the methionine-sulfonium phenyl substrate of claim 1, in the active site is identified through the following steps:
a) Obtaining a plurality of fluorinase protein sequences from a non-redundant database;
b) Modeling the obtained fluorinase protein sequences and achieving maximum 3D fitting of the active site with a reference active site that contains a specific F⁻ ion attack conformation against the modeled the methionine-sulfonium phenyl substrate of claim 1. Transforming the coordinates of the F⁻ ion and the substrate into the newly modeled fluorinase to facilitate their interaction within the active site; and
c) Subjecting the newly modeled fluorinase to a screening protocol that includes metadynamics simulations and free energy surface calculations to identify the most suitable fluorinase enzyme demonstrating stable catalytic binding of the methionine-sulfonium phenyl substrate of claim 1 in the active site.

3. The method of claim 2, wherein the selected fluorinase enzyme incorporates specific mutations to optimize the binding affinity of the methionine-sulfonium phenyl substrate.

4. An Engineered fluorinase polypeptide of claim 3, having fluorination activity comprises an amino acid sequence that is at least 75% identical to SEQ ID NO: 2 and that includes the feature of residue corresponding to X143 is W, and X151 is Y.

5. The engineered fluorinase polypeptide of claim 4 comprises an amino acid sequence given by SEQ ID NO: 3, 4, 5 and 6 wherein the amino acid sequence additionally includes at least one or more of the following features:
a) Residue corresponding to X38 is Aspartic acid or is a Polar, charged, aliphatic or aromatic residue or
b) Residue corresponding to X39 is Isoleucine or an Aliphatic or polar residue or
c) Residue corresponding to X43 is Serine or Polar, charged, or aliphatic residue or
d) Residue corresponding to X45 is Leucine or Polar, charged, aliphatic or aromatic residue or
e) Residue corresponding to X63 is Serine or a non-polar or aliphatic residue or
f) Residue corresponding to X65 is Arginine or a non-polar or aliphatic residue or
g) Residue corresponding to X156 is Isoleucine or an aliphatic residue or
h) Residue corresponding to X195 is Threonine or Polar, charged, aliphatic or aromatic residue.
